(19) United States
(12) Patent Application Publication
(10) Pub. No.: US 2011/0313039 A1
(43) Pub. Date: Dec. 22, 2011

(54) METHOD FOR INCREASING ENDOGENOUS PLASMALOGEN LEVELS IN MAMMALS

(75) Inventors: Frederic Destaillats, Servion (CH); Jean-Baptiste Bezelgues, Powell, OH (US); Fabiola Dionisi, Epalinges (CH); Cristina Cruz-Hernandez, Epalinges (CH); Isabelle Masserey-Elmelegy, Epalinges (CH)

(73) Assignee: NESTEC S.A., Vevey (CH)

(21) Appl. No.: 13/254,622

(22) PCT Filed: Feb. 24, 2010
(86) PCT No.: PCT/EP10/52305

§ 371 Date: Sep. 2, 2011

(30) Foreign Application Priority Data
Mar. 4, 2009 (EP) 09154308.2

(51) Int. Cl. A61K 31/225; A61P 3/00; A61P 25/28; A61K 31/08; A61P 35/00

(52) U.S. Cl. 514/547; 514/723

(57) ABSTRACT

The present invention relates generally to compounds such as alkylglycerol and alkoxyglycerol for use in increasing the endogenous level of plasmalogens in a mammal. In particular, these compounds are used for increasing the endogenous plasmalogen level to a level greater than the level in healthy mammals. According to the present invention such compounds are also for use in the prevention or treatment of diseases caused or affected by a decreased endogenous level of plasmalogens. A method for the manufacture of a dietary precursor for the use of the present invention is also part of the present disclosure.

### FIELD OF THE INVENTION

The present invention relates generally to compounds such as alkylglycerol and alkoxyglycerol for use in increasing the endogenous level of plasmalogens in a mammal. In particular, these compounds are used for increasing the endogenous plasmalogen level to a level greater than the level in healthy mammals. According to the present invention such compounds are also for use in the prevention or treatment of diseases caused or affected by a decreased endogenous level of plasmalogens. A method for the manufacture of a dietary precursor for the use of the present invention is also part of the present disclosure.

### BACKGROUND ART

Plasmalogens and their structure and uses are known to skilled artisans.

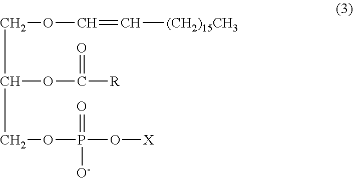

Plasmalogens are glycerol ether phospholipids wherein a glycerol moiety is bound to a 1-alkenyl ether group or a 1-alkyl ether group. Three major classes of plasmalogens have been identified and designated choline, ethanolamine, and serine plasmalogens. The chemical structure of one group of plasmalogens is shown in (3), which illustrates the ether linkage at Cl on the glycerol backbone. Typically, R is a hydrocarbon chain of varying length and X is choline, ethanolamine, or serine.

Plasmalogens are known to be associated with various diseases and conditions in animals, particularly in animals with low endogenous plasmalogen levels. Similarly, endogenous plasmalogen levels are known to decrease as an animal ages, possibly resulting in the onset of diseases and conditions adverse to the animal's health. It is not clear if these variations in plasmalogen levels are due to a high turnover of plasmalogen or a decrease in enzymatic peroxisomal activities which are essential for the biosynthesis of plasmalogens.

Their suggested functions include protection against oxidative stress, participation in signal transduction, membrane fusion events, cholesterol transport and membrane trafficking, processes known to be disturbed in sphingolipidoses (cf. Nagan, N. et al., in Prog. Lipid Res. 10, 2001, 199-229 and Gorgas, K. et al. in BBA 1763, 2006, 1511-1526).

The level of plasmalogens, which is often measured in erythrocytes, was found to be low in severe diseases such as dementia, Alzheimer's by Goodenowe, D. B. et al. in J. Lipid Res., 48, 2007, 2485-2498. Moraitou M. et al. in Blood Cells Mol. Dis. 2008, also found low levels of plasmalogens in Gaucher disease. It is also suggested that plasmalogen status is impaired in metabolic syndrome and other chronic diseases.

U.S. Pat. No. 5,759,585 discloses the use of plasmalogens for treating neurodegenerative diseases. U.S. Pat. No. 6,177,476 discloses methods for replenishing plasmalogens in mammals using monoethers of glycerols and their carboxylic acid ester derivatives. WO 08/124916 Al discloses methods for the diagnosis and risk assessment of plasmalogen deficiency mediated diseases of aging, particularly colon cancer, prostate cancer, lung cancer, breast cancer, ovarian cancer, kidney cancer, cognitive impairment, and dementia.

Given the adverse effect of low endogenous plasmalogen levels on animals and their health, there is thus a need for ways to easily improve the endogenous plasmalogen level in mammals.

### SUMMARY OF THE INVENTION

The object of the present invention is solved by means of the independent claims. The dependent claims further develop the central idea of the invention.

Thus, in a first aspect, the present invention relates to compound selected from the group of alkylglycerols (1) and/or alkoxyglycerols (2)

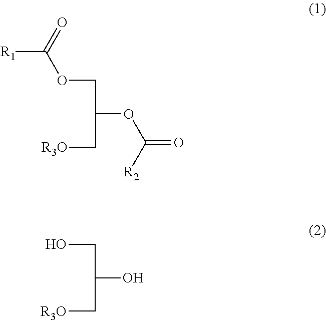

wherein R₁, R₂ and R₃ are alkyl chains, for use in increasing the endogenous level of plasmalogens in a mammal to a level greater than the level of plasmalogens in said healthy mammal.

A second aspect of the invention pertains to compound selected from the group of alkylglycerols having formula (1) wherein R₁ and R₂ are alkyl chains having more than 8 carbons, and R₃ is a saturated or unsaturated alkyl chain, preferably wherein R₃ comprises 18 carbons and one ethylenic double bond, more preferably wherein R₃ is a 9-octadecenyl group, even more preferably wherein is a (Z)-9-octadecenyl group and/or alkoxyglycerols having formula (2) wherein R₃ is a saturated or unsaturated alkyl chain, preferably wherein R₃ comprises 18 carbons and one ethylenic double bond, more preferably wherein R₃ is a 9-octadecenyl group, even more preferably wherein is a (Z)-9-octadecenyl group

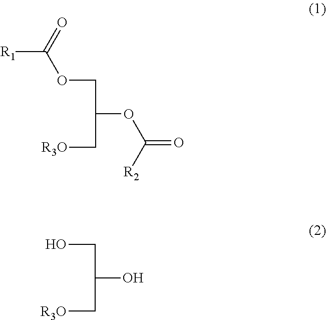

for use in preventing or treating a disease or condition in a mammal caused or affected by decreased plasmalogen levels such as metabolic syndrome, neurodegenerative disease, dementia, Alzheimer's disease, cognitive impairment, colon cancer, prostate cancer, lung cancer, breast cancer, ovarian cancer, and kidney cancer.

The present invention also extends to the use of the compounds described in the present invention for the preparation of a composition to treat or prevent one or more of the disorders mentioned herein.

Such compositions may be selected from the group consisting of food products, animal food products, drinks, nutraceuticals, food additives, nutritional compositions, pharmaceutical compositions and/or medicaments.

Finally, a method for the manufacture of a dietary precursor for increasing the endogenous level of plasmalogens in a mammal to a level greater than the level of plasmalogens in said healthy mammal comprising the steps of:
a. Providing a compound selected from alkylglycerols having formula (1) wherein R₁ and R₂ are alkyl chains having more than 8 carbons, and R₃ is a saturated or unsaturated alkyl chain, preferably wherein R₃ comprises 18 carbons and has one ethylenic bond, more preferably wherein R₃ is a 9-octadecenyl group, even more preferably wherein is a (Z)-9-octadecenyl group
and/or alkoxyglycerols having formula (2) wherein R₃ is a saturated or unsaturated alkyl chain, preferably wherein R₃ has 18 carbons and one ethylenic bond, more preferably wherein R₃ is a 9-octadecenyl group, even more preferably wherein is a (Z)-9-octadecenyl group

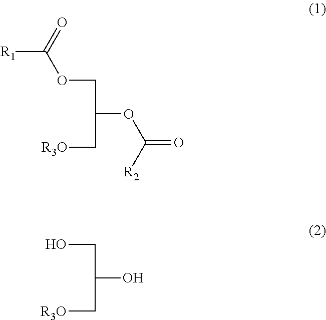

b. Mixing said compound with further nutritional ingredients, preferably selected from the group of proteins, peptides, carbohydrates, lipids, minerals, vitamins, probiotics or any mixtures thereof, to give said dietary precursor
   is also part of the present invention.

### FIGURES

The present invention is described further herein by reference to the accompanying figures in which

FIG. 1 is a gas-liquid chromatography analysis of the saponified fraction (TMS derivatives) of shark liver oil showing the occurrence of sn-1-alkoxyglycerols with mainly an 18:1 alkoxy group;

FIG. 2 shows the relative level (% of total fatty acid methyl ester (FAME)) of (A) total dimethyl acetate (DMA), (B) DMA (18:1), (C) DMA (18:0) and (D) DMA (16:0) in astrocytes incubated for 24 h with alkoxyglycerol obtained by saponification of shark liver oil or the culture media (control group).

### DETAILED DESCRIPTION OF THE INVENTION

The present invention relates to compounds selected from the group of alkylglycerols and/or alkoxyglycerols.

By “alkylglycerol” is meant a compound having structure (1), wherein R₁, R₂ and R₃ are alkyl chains.

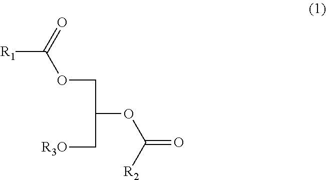

By “alkoxyglycerol” is meant a compound having structure (2), wherein R₃ is an alkyl chain.

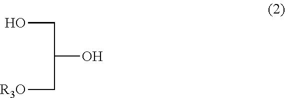

All optical isomers, mixtures thereof, racemates thereof, enantiomerically enriched and purified forms of the compounds of formula (1) or (2) are encompassed by the depicted structures (1) and (2).

These compounds have been found by the present inventors to exhibit excellent activity on the plasmalogen level of mammals. This in vivo effect of the alkylglycerol and/or alkoxyglycerol is further unexpected since such compounds have only been reported to replenish the level of plasmalogen to a normal state from a diseased state.

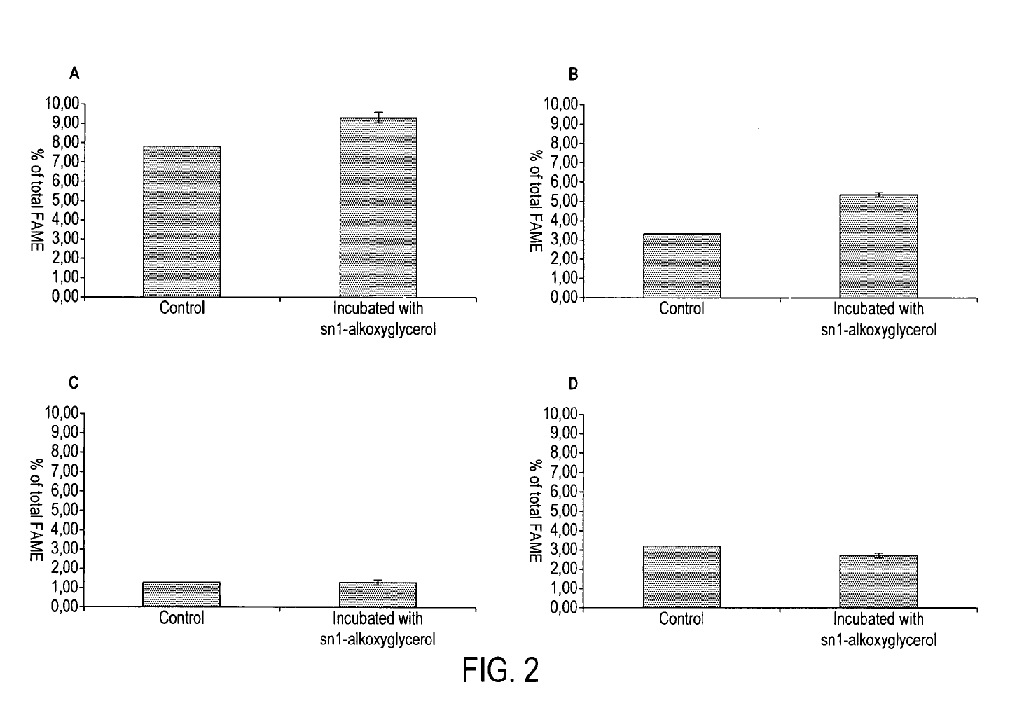

In the present invention, compounds selected from the group of alkylglycerol and/or alkoxyglycerol have been found to increase the endogenous level of plasmalogens in a mammal to a level greater than the endogenous level of plasmalogens in said healthy mammal. This is illustrated in FIG. 2 which shows that the total amount of dimethylacetate (DMA) upon incubation with a saponified fraction of shark liver oil which contains alkoxyglycerol according to the present invention is increased compared to a control. Additionally example 2 also shows a noticeable increase on the plasmalogen level when using alkylglycerol according to the present invention.

In the present invention, the mammal may be a human or a companion animal such as a dog or a cat. The mammal may be an aging mammal. By “aging” is meant that the mammal has exceeded 50% of the average lifespan for its particular species and/or breed within a species.

The endogenous level of plasmalogens in a mammal can be estimated by a method which measures the level of dimethylacetate (DMA) in astrocytes. DMAs are specifically formed from vinyl-ether chain and are usually used to assess the level of vinyl-ether lipids such as plasmalogens. Astrocytes are selected as an in vitro model since this glial cell type is a central element of brain lipid metabolism.

Using such a method, it was found that the level of plasmalogen is increased when a healthy mammal is administered with an alkylglycerol and/or an alkoxyglycerol compound of the invention. In a preferred embodiment, the endogenous level of plasmalogens is increased by at least 5%, preferably by at least 10%, more preferably by at least 15% when compared to the endogenous level of a healthy mammal.

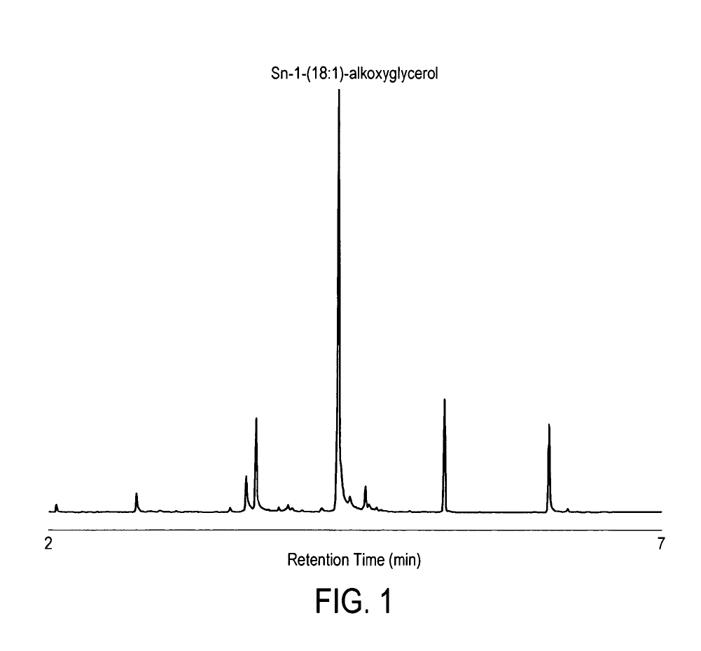

The alkylglycerol and/or alkoxyglycerol compounds used in the present invention are preferably derived from natural biomass such as animal products, microorganisms, natural products. More preferably, the compounds are derived from marine oil such as fish oils and/or egg lecithins. In a most preferred embodiment, the compounds are derived from shark liver oil. The compounds derived from shark liver oil have been shown to be particularly effective in increasing the endogenous level of plasmalogens in a mammal. The compounds derived from shark liver oil may vary in their composition. For example, FIG. 1 shows the gas chromatography trace of a saponified fraction of shark liver oil showing the occurrence of a main fraction of alkoxyglycerols having a 18:1 alkoxy group.

In a preferred embodiment, the alkylglycerol is a compound of formula (1) wherein R₁ and R₂ are alkyl chains having more than 8 carbons, and R₃ is a saturated or unsaturated alkyl chain. Preferably, R₃ comprises 18 carbons and one ethylenic double bond, more preferably R₃ is a 9-octadecenyl group, even more preferably R₃ is a (Z)-9-octadecenyl group.

In a further embodiment, the alkoxyglycerol is a compound of formula (2) wherein R₃ is a saturated or unsaturated alkyl chain, preferably wherein R₃ has 18 carbons and one ethylenic bond, more preferably wherein R₃ is a 9-octadecenyl group, even more preferably wherein R₃ is a (Z)-9-octadecenyl group.

The alkylglycerol and alkoxyglycerol of the present invention may be used in combination or separately.

The compounds of formula (1) and/or (2) may be used in an amount of 1-1000 mg/kg/day. By mg/kg/day is meant the amount of compound (in mg) per kg of weight of the mammal per day. Thus, for example, a pet weighing 20kg will require 20-20000 mg of compound per day. Preferably, the compounds are used in an amount of 5-500, more preferably 10-300 mg/kg/day.

The compounds used in the present invention may be formulated for oral or topical application. Preferably, the compounds are formulated as an oil or fat, a capsule, a tablet, a powder, a syrup, a liquid or semi-liquid.

In a preferred embodiment, the compound is used as a nutritional supplement or is incorporated into food products. When incorporated into food products, it is preferably in an amount of 1-1000, more preferably 5-500, even more preferably 10-300 mg/kg of diet. By “kg of diet” is meant the amount of food in kg consumed by the mammal.

The food products to which the compounds of the invention may be incorporated may be selected from beverages, instant beverages, culinary products, frozen foods, dairy products, confectionery products, wet and dry petfood.

Due to their ability to increase the endogenous level of plasmalogen in a mammal to a level greater than the level in said healthy mammal, the compounds of the invention are also for use in preventing or treating a disease or condition in a mammal caused or affected by decreased plasmalogen levels such as metabolic syndrome, neurodegenerative disease, dementia, Alzheimer's disease, cognitive impairment, colon cancer, prostate cancer, lung cancer, breast cancer, ovarian cancer, and kidney cancer.

A particular advantage of the invention is the preventative effect of the compounds which hitherto were only considered to merely improve a diseased state.

A method for the manufacture of a dietary precursor for increasing the endogenous level of plasmalogens in a mammal to a level greater than the level of plasmalogens in said healthy mammal also forms part of the present invention.

The first step in the method is providing a compound selected from alkylglycerols of formula (1) wherein R₁ and R₂ are alkyl chains having more than 8 carbons, and R₃ is a saturated or unsaturated alkyl chain, preferably wherein R₃ comprises 18 carbons and has one ethylenic bond, more preferably wherein R₃ is a 9-octadecenyl group, even more preferably wherein R₃ is a (Z)-9-octadecenyl group and/or alkoxyglycerols of formula (2) wherein R₃ is a saturated or unsaturated alkyl chain, preferably wherein R₃ has 18 carbons and one ethylenic bond, more preferably wherein R₃ is a 9-octadecenyl group, even more preferably wherein R₃ is a (Z)-9-octadecenyl group.

Providing such compounds may be done by isolation from a natural biomass, preferably from a marine oil such as fish oils. More preferably the compounds are provided by isolation from shark liver oil. Such isolation methods are known to a skilled person.

Alternatively, the compounds may be provided by synthesis such as chemical synthesis, or by biotransformation using microorganisms and/or enzymes for example. These methods are known to a skilled person. For example, obtaining the compounds by biotransformation may involve the steps of selection of the microorganism, fermentation, purification of the alkylglycerol or alkoxyglycerol fractions.

Once the compounds have been provided, they are mixed with further nutritional ingredients in a second step of the method. Such nutritional ingredients may be selected from the group of proteins, peptides, carbohydrates, lipids, minerals, vitamins, probiotics or any mixtures thereof.

The mixture may then be further processed by methods known to a skilled person or used as such as a dietary precursor.

The dietary precursor obtained by the method of the present invention preferably comprises 0.1-100, more preferably 1-40, even more preferably 3-10 wt % of said alkylglycerol and/or alkoxyglycerols.

The dietary precursor may be a complete nutritional product such as beverages, instant beverages, culinary products, frozen foods, dairy products, confectionery products, wet and dry petfood etc. Alternatively, the dietary precursor may be formulated as an oil or fat, a capsule, a tablet, a powder, a syrup, a liquid or semi-liquid which may be incorporated into a mammal's diet.

The present invention is further illustrated by means of the following non-limiting examples.

### EXAMPLES

### Example 1

A. Preparation of Alkoxyglycerol Fraction from Shark Liver Oil Alkylglycerol

Alkoxyglycerol fraction has been prepared from squalene free shark liver oil by saponification according to the following procedure. Alkoxyglycerols were obtained by saponification of 100 g of desqualenised shark liver oil with 1000 ml potassium hydroxyde (1M) ethanolic solution. The resulting solution which contains the alkoxyglycerol was then extracted with diethylether (3×300 mL) followed by a distillation step. The resulting white waxy residue contained around 90% of alkoxyglycerols.

B. Incubation of Alkoxyglycerol with Astrocytes

Astrocytes have been selected as an in vitro model because this glial cell type is a central element of brain lipid metabolism. Briefly, the saponified shark liver oil (100 μM) has been added to astrocytes for 24 h. After the treatment period, the cells has been scrapped in methanol and directly derivatised prior to analysis. Non-supplemented cells were used as control and the level of dimethylacetate (DMA) derivatives have been measured by gas-liquid chromatography. DMAs are specifically formed from vinyl-ether chain and are usually used to assess the level of vinyl-ether lipids such as plasmalogens. Three main plasmalogen derived DMA which are DMA(16:0), DMA(18:0) and DMA(18:1) have been detected in the control cells. The analysis of astrocytes incubated for 24 h revealed a substantial increase of the total DMA and DMA 18:1 as described in Table 1.

TABLE 1
Plasmalogen level measured as DMA in astrocytes incubated with
alkoxyglycerols compared to control (cell culture medium)

| | | DMA | DMA | DMA | Total |
|---|---|---|---|---|---|
| | | 16:0 | 18:0 | 18:1 | DMA |
| Control | Mean | 3.17 | 1.25 | 3.36 | 7.79 |
| | SD | 0.16 | 0.06 | 0.17 | 0.39 |
| Incubated with sn1- | Mean | 2.73 | 1.31 | 5.34 | 9.38 |
| alkoxyglycerol | SD | 0.09 | 0.14 | 0.11 | 0.27 |

C. Conclusion

This example shows that alkoxyglycerols prepared from alkylglycerols can be efficiently used as plasmalogen precursors by neural cells such as astrocytes.

### Example 2

A. Animal Study

Adult male Sprague Dawley rats (4 weeks of age, Centre d'élevage Janvier, Le Genest Saint Isle, France) were fed for 3 weeks with either a control diet, a diet enriched with alkylglycerols (corresponding to a consumption of 300 mg of alkylglycerol per day, 340 μmol/day) or a diet enriched with alkoxyglycerols (corresponding to a consumption of 116 mg of alkoxyglycerols per day, 340 μmol/day) (n=6 per group). All three diets were similar in term of composition: 23% of saturated fatty acids, 60% of monounsaturated fatty acids, 13% of linoleic acid, 1.3% of α-linolenic acid, 1.8% of docosahexaenoic acid.

B. Results

The level of plasmalogen measured as total DMA was found to be significantly higher in the animal receiving the alkylglycerol supplemented diet. The supplementation of the diet with the alkoxyglycerol supplemented diet also induced an increase of the plasmalogen level.

TABLE 2
Plasmalogen level measured as DMA in red blood cell lipids
from male rats fed 21 days with a diet enriched in
alkylglycerols or alkoxyglycerols, compared to a contol diet.

| | | |
|---|---|---|
| | Alkylglycerol | Alkoxyglycerol |
| Control diet | diet | diet |

| | Mean | SD | Mean | SD | Mean | SD |
|---|---|---|---|---|---|---|
| DMA | 6.61 | 0.55 | 7.89 | 0.33 | 7.02 | 0.52 |

C. Conclusion

Supplementation of diet with alkylglycerol and alkoxyglycerol was shown to be effective to increase plasmalogen level in red blood cells in animal model.

1. Compound A method for increasing the endogenous levels of plasmalogens in a mammal to a level greater than the endogenous level of plasmalogens in the healthy mammal comprising the step of administering a composition comprising a compound selected from the group consisting of alkylglycerols (1) and/or alkoxyglycerols (2) 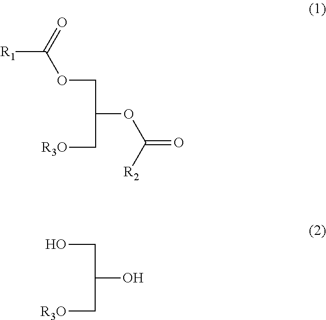
wherein R₁, R₂ and R₃ are alkyl chains to the mammal.

2. Method according to claim 1, wherein the compound is derived from natural biomass.

3. Method according to claim 1, wherein the alkylglycerol is a compound of formula (1) wherein R₁ and R₂ are alkyl chains having more than 8 carbons, and R₃ is a saturated or unsaturated alkyl chain.

4. Method according to claim 1, wherein R₃ comprises 18 carbons and one ethylenic double bond.

5. Method according to claim 1, wherein the alkoxyglycerol is a compound the formula (2) wherein R₃ is a saturated or unsaturated alkyl chain.

6. Method according to claim 1, wherein the mammal is selected from the group consisting of a human and a companion animal.

7. Method according to claim 1, wherein the endogenous level of plasmalogens is increased by at least 5% compared to the level of plasmalogens in the healthy mammal.

8. Method according to claim 1, wherein the composition is used in an amount of 1-1000, mg/kg/day.

9. Method according to claim 1, wherein the composition is for oral or topical application.

10. Method according to claim 1, wherein the composition is a nutritional supplement.

11. Method according to claim 1, wherein the composition is selected from the group consisting of beverages, instant beverages, culinary products, frozen foods, dairy products, confectionery products, wet and dry petfood.

12. Method for preventing or treating a disease or condition in a mammal caused or affected by decreased plasmalogen levels comprising the step of administering a composition including a compound selected from the group consisting of alkylglycerols of formula (1) wherein R₁ and R₂ are alkyl chains having more than 8 carbons, and R₃ is a saturated or unsaturated alkyl chain, and/or alkoxyglycerols of formula (2) wherein R₃ is a saturated or unsaturated alkyl chain to a mammal in need of same.

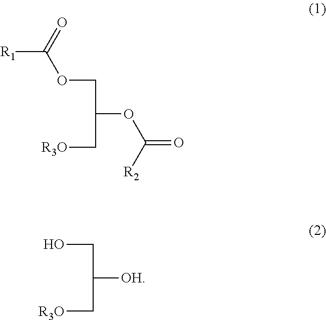

13. Method for the manufacture of a dietary precursor for increasing the endogenous level of plasmalogens in a mammal to a level greater than the level of plasmalogens in said healthy mammal comprising the steps of:
providing a compound selected from the group of alkylglycerols of formula (1) wherein R₁ and R₂ are alkyl chains having more than 8 carbons, and R₃ is a saturated or unsaturated alkyl chain.
and alkoxyglycerols of formula (2) wherein R₃ is a saturated or unsaturated alkyl chain. 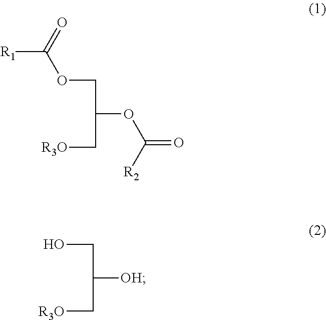
and
mixing the compound with further nutritional ingredients, preferably selected from the group consisting of proteins, peptides, carbohydrates, lipids, minerals, vitamins, probiotics and mixture.

14. Method according to claim 13, wherein the alkylglycerol and/or alkoxyglycerol are provided by isolation from a natural biomass.

15. Method according to claim 13, wherein the dietary precursor comprises 0.1-100.

16. Method according to claim 12, wherein the decreased plasmalogen levels is caused by a disease selected from the group consisting of metabolic syndrome, neurodenerative disease, dementia, Alzheimer's disease, cognitive impairment, colon cancer, prostate cancer, lung cancer, breast cancer, ovarian cancer, and kidney cancer.
